(19) United States
(12) Patent Application Publication
(10) Pub. No.: US 2019/0328319 A1
(43) Pub. Date: Oct. 31, 2019

(54) SYSTEM AND METHOD FOR DETERMINING A PERSONALIZED ANTI-SNORING SOLUTION

(71) Applicant: KONINKLIJKE PHILIPS N.V., EINDHOVEN (NL)

(72) Inventors: Koray KARAKAYA, Eindhoven (NL); Stefan WINTER, Würselen (DE); Anastasia SCHMALZ, Amsterdam (NL)

(73) Assignee: KONINKLIJKE PHILIPS N.V., EINDHOVEN (NL)

(21) Appl. No.: 16/473,403

(22) PCT Filed: Dec. 27, 2017
(86) PCT No.: PCT/EP2017/084612

§ 371 Date: Jun. 25, 2019

Related U.S. Application Data
(60) Provisional application No. 62/439,909, filed on Dec. 29, 2016.

(51) Int. Cl. A61B 5/00; A61B 7/00

(52) U.S. Cl. CPC A61B 5/4815; A61B 5/7264; A61B 7/003

(57) ABSTRACT

A method for determining a personalized anti-snoring solution for a patient includes performing an analysis of a sleep period of the patient in which snoring by the patient is occurring; advising the patient of an anti-snoring solution based on information obtained in the analysis; tracking a further sleep period of the patient in which the patient is employing the anti-snoring solution; and advising the patient of changes to the anti-snoring solution based on a comparison of information obtained in the tracking with data obtained in the analysis.

### BACKGROUND OF THE INVENTION

### 1. Field of the Invention

The invention pertains to methods for determining a personalized anti-snoring solution for a patient. The invention further pertains to systems for use in determining a personalized anti-snoring solution for a patient.

### 2. Description of the Related Art

Snoring is a concern for those suffering from the condition as snoring can result in low quality sleep and frequent awakenings. Snoring is also not only a concern for snorers themselves, but also a major source of annoyance for the bed partners of those who frequently snore. Hence snoring has a significant effect on the sleep quality of both the snorer and the bed partner.

Despite there being a large snoring population, the amount of snorers who can find an efficient and acceptable solution to their snoring problem is very limited. In a recent study we have observed that more than 90% of snorers who have tried a snoring solution are not happy with the solutions they have been using. A majority of them also report that the anti-snoring devices they have tried are not effective. There are different anti-snoring device classes commonly used to treat snoring. Mandibular advancement devices (MADs), position correction devices, and nasal dilators are the three most common device classes. The device portfolio further expands to other possible solutions such as: position correction trainers, pillows and mattresses, chin straps, etc. All of the aforementioned devices have a certain level of success rate for minimizing snoring, for different types of snoring.

The source of the snoring sound can be various: originated from the soft palate and tissue in the mouth, nose or throat vibrating. The source location may vary for different snorers and is related to the physiology of the upper airway, which may be deformed during sleep. Different types of anti-snoring devices are more effective for dealing with different type of source location (e.g., a nasal dilator is more effective for snoring originated from nasal obstructions, a position correction device helps dealing with the positional (gravitational) collapse of the soft tissue, etc.). Therefore, it is essential to use have the right type of device for an effective solution.

However, it is a problem for a habitual snorer to find the most effective device. As a result, habitual snorers tend to choose devices not based on their expected effectiveness, but for other factors such as cost, ease of use, etc. Moreover, correct and comfortable use of these devices, without compromising performance of the devices is yet another challenge.

Such challenges also show themselves in existing business models around anti-snoring devices as different device classes (e.g. nasal dilators, mandibular advancement devices and position correction devices), coming from different brands and manufacturers, are sold individually. Accordingly, it is a rather scattered domain and makes it even more difficult for snorers to navigate through the device selection process, as there is no “one-stop shop solution” for sleep disordered breathing problems, particularly for snoring.

### SUMMARY OF THE INVENTION

In one embodiment, a method for determining a personalized anti-snoring solution for a patient is provided. The method comprises: performing an analysis of a sleep period of the patient in which snoring by the patient is occurring; advising the patient of an anti-snoring solution based on information obtained in the analysis; tracking a further sleep period of the patient in which the patient is employing the anti-snoring solution; and advising the patient of changes to the anti-snoring solution based on a comparison of information obtained in the tracking with data obtained in the analysis.

Analyzing snoring by the patient may comprise performing an acoustic analysis of the snoring by the patient.

Performing an acoustic analysis of the snoring by the patient may comprise one or more of: extracting snoring episodes from within a mixed acoustic signal; classifying snoring intensity of the snoring episodes; calculating the proportion of snoring episodes overnight; classifying the snoring episodes sound amplitude; and storing data resulting from the acoustic analysis.

Analyzing snoring by the patient may comprise performing a spectral analysis of the snoring by the patient.

Analyzing snoring by the patient may comprise storing data related to the sound of the snoring by the patient in a snoring sound library.

Analyzing snoring by the patient may comprise performing a subjective assessment of snoring behavior by the patient.

Performing a subjective assessment may comprise providing a number of questions to one or both of the patient and a bed partner of the patient and analyzing responses thereto.

Analyzing snoring by the patient may comprise performing a self-assessment for determining a source of the snoring by the patient.

Performing a self-assessment may comprise generating snoring sounds by closing and opening nostrils and mouth of the patient in order to identify the most likely source of the snoring by the patient.

In another embodiment, a computer program product is provided which includes a non-transitory computer readable medium encoded with a computer program comprising program code for implementing the method or methods described herein.

In yet another embodiment, a system for determining a personalized anti-snoring solution for a patient is provided. The system comprises: a processing device having a suitable memory associated therewith; one or more acoustic sensors in communication with the processing device; and an input/out device structured to receive information from and provide information to the patient. The processing device is programmed to: utilize the one or more acoustic sensors in performing an analysis of snoring by the patient; utilize the input/output device in advising the patient of an anti-snoring solution based on information obtained in the analysis; utilize the one or more sensors in tracking further snoring, by the patient in response to the patient employing the anti-snoring solution; and utilize the input/output device in advising the patient of changes to the anti-snoring solution based on a comparison of information obtained in the tracking with data obtained in the analysis.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a flow diagram of a method according to one example embodiment of the present invention; and

FIG. 2 is a schematic diagram of a system according to one example embodiment of the present invention.

### DETAILED DESCRIPTION OF EXEMPLARY EMBODIMENTS

As used herein, the singular form of “a”, “an”, and “the” include plural references unless the context clearly dictates otherwise. As employed herein, the term “number” shall mean one or an integer greater than one (i.e., a plurality).

As used herein, “snoring” shall refer to the undesired occurrence of vibration of respiratory structures and the resulting sound due to obstructed air movement in a user during breathing while sleeping.

As used herein, “patient” shall be used to refer to a person in which snoring occurs.

As used herein, “bed partner” shall be used to refer to a person who sleeps with or nearby a patient.

As used herein, the term “controller” or “processing device” shall mean a programmable analog and/or digital device (including an associated memory part or portion) that can store, retrieve, execute and process data (e.g., software routines and/or information used by such routines), including, without limitation, a field programmable gate array (FPGA), a complex programmable logic device (CPLD), a programmable system on a chip (PSOC), an application specific integrated circuit (ASIC), a microprocessor, a microcontroller, a programmable logic controller, or any other suitable processing device or apparatus. The memory portion can be any one or more of a variety of types of internal and/or external storage media such as, without limitation, RAM, ROM, EPROM(s), EEPROM(s), FLASH, and the like that provide a storage register, i.e., a non-transitory machine readable medium, for data and program code storage such as in the fashion of an internal storage area of a computer, and can be volatile memory or nonvolatile memory.

As described above, finding the right anti-snoring solution is difficult for snorers as they are typically not aware of what type of snoring problem they have (e.g. nasal, palatal, positional, seasonal, etc.). Moreover, snorers generally do not associate different types of anti-snoring solutions with different devices. These technical and physiological challenges, combined with the complexity of the anti-snoring device market, result in a difficult decision journey for snorers and for their bed partners. Embodiments of the invention address these problems by providing a system and method which identifies a personalized anti-snoring solution for a patient while also ensuring the patient is using it in an effective manner.

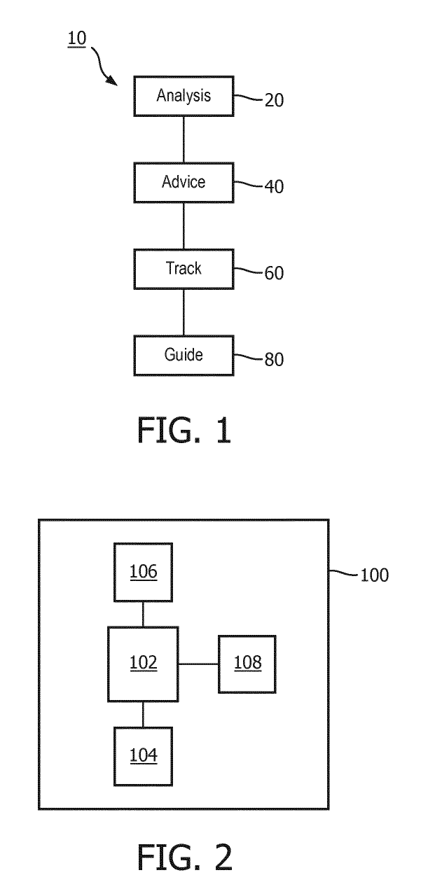

FIG. 1 illustrates a flow diagram of a method 10 for determining a personalized anti-snoring solution for a patient according to an example embodiment of the invention. Method 10 includes four basic steps: analysis 20, advice 40, track 60 and guide 80.

Beginning with the analysis step 20, the snoring behavior and pattern of the user is analyzed over a time period. This time period is also used as a baseline for comparing with subsequent time period (discussed below) to determine changes which may occur to the user's snoring behavior and pattern as a result of employing an anti-snoring solution as described herein. As part of the analysis step 20, an acoustic analysis of the snoring prevalence (e.g., total snoring time per night, snoring sound amplitude, etc.) is carried out. Such acoustic analysis includes: extracting snoring episodes from within a mixed acoustic signal that may consist of various background noises; classifying snoring intensity (e.g. low, loud, very loud); calculating the proportion of snoring episodes overnight, and (optionally) classifying them by sound amplitude; and storing resulting data from the period of time to serve as a baseline for later comparison to show progress/trends of snoring prevalence on a weekly, monthly, or longer term.

As another part of the analysis step 20, a spectral analysis of the sound of snoring by the patient is carried out using a means of spectral analysis (e.g., fast Fourier transform—FFT, linear predictive coding—LPC, etc.) for resolving the spectral and psychoacoustic features of the snoring sounds. Such features are used for: building a snoring sound library in which data related to the snoring sound of the user is stored for later use; assessing the link between anti-snoring devices and the changes in the spectral profile (e.g. changes of a certain frequency band as a result of using an anti-snoring device); and assessing the link between the snoring source (e.g. back of the throat, palate, nose, etc.) and the frequency spectrum.

As another part of the analysis step 20, a subjective assessment of snoring behavior of the patient may be conducted. Such subjective assessment may include input from the patient and/or the bed partner of the patient (where applicable), and/or someone who can observe the snoring behavior of the snorer (e.g. via simple observation or an observation carried out via audio/video recordings). The subjective assessment may be carried out, for example, without limitation, through the use of questionnaires, in paper, electronic, or any other suitable form which include a number of questions pertaining to various aspects related to situations in which snoring by the patient occurs (e.g. “do you observe your partner snoring louder/more frequent while sleeping on his/her back?”, “do you snore every day?”, “do you snore louder on some days than others?”, “have you observed a link between your snoring behavior and external factors like nasal congestion, alcohol intake, etc.?”).

As yet another part of the analysis step 20, a self-assessment may be carried out in order to attempt to locate the source of snoring in the patient. Such self-assessment focuses on physiological aspects of snoring that help identify the source (e.g. positional, nasal, palatal, etc.) of snoring and may include steps such as generating snoring sounds by closing and opening nostrils and mouth of the patient in order to identify the most likely source of snoring.

Accordingly, it is to be appreciated that the analysis step 20 is utilized to generally gather information/data regarding snoring by the patient for a number of further uses. Such information/data may be gathered, processed, stored, etc. as needed, by a suitable processing device such as, for example, without limitation, a computer or a smartphone.

As will be discussed further below, one such use of the information gathered in analysis step 20 is as a baseline for comparing changes in snoring by a user to determine effectiveness of anti-snoring solutions implemented by the user. Another use of such information/data obtained in step 20 occurs in advice step 40 wherein the patient is advised of potential anti-snoring solutions which may reduce and/or inhibit snoring or attributes thereof (occurrence, intensity, etc.).

Advice step 40 includes suggesting an appropriate anti-snoring solution, or a combination thereof based on the data gathered in the analysis step. Anti-snoring solutions are clustered for their effective mechanism; e.g. nasal dilator, position correction device(s)/position correction trainer, a mandibular advancement device, etc. As such devices are known to those skilled in the art, a detailed description of such devices is not provided herein. As an example, if from the information gathered in step 20 it is found that snoring is more prominent while the person sleeps on his/her back, then a positional correction device would be advised. Yet another example would be on the nasal obstruction vs. nasal dilator; if the patient is not qualified for these two options, then the first advice would be using a MAD. In addition, or in some situations as an alternative, a suggested habit change or changes (e.g., without limitation, weight loss, minimizing alcohol intake during night, using air purifier/humidifier for minimizing nasal obstructions, especially during the allergy season, giving generic advice on indoor allergens that causes nasal congestion/allergic rhinitis, etc.) may be provided to the patient.

After the patient receives one or more anti-snoring solutions at step 40, the patient's snoring subsequent to implementation of the one or more anti-snoring solutions is tracked in track step 60. Track step 60 is generally a derivative of the analysis step 20 previously described, and as such may include any or all of the steps previously discussed in regard to step 20. Tracking step 60 may be carried out by collated representation of daily (nightly) analysis of the user's snoring (or lack thereof) over a desired period of time in order to obtain a satisfactory representation of the effect of the anti-snoring solution or solutions advised in step 40. An important aspect of the tracking step 60 is coupling the analysis results to use of an anti-snoring device. For example, as previously discussed, during the analysis step 20 a baseline about how the patient snores is created, such baseline provides the opportunity to then build a comparison between two states: patient sleep without any anti-snoring device and with an anti-snoring device.

A secondary use of the tracking step 60 is establishing the links between the snoring behavior with other factors such as, for example, without limitation, alcohol intake, sleep position, bedroom conditions (climate, air quality, noise, light intensity, etc.), bed partner's sleeping behavior, etc. The use of further questionnaires in different forms is a good way for accomplishing this, especially in regard to alcohol intake. However, data from different sensors such as bedroom conditions, and also sleep position (e.g. by using an inclinometer attached to the patient's cloths) may also be utilized. The same applies to the bed partner's sleeping behavior as well: a subjective survey and/or the input from a sleep quality monitoring sensor could be used to provide this information.

After sufficient information/data has been gathered in track step 60, the information/data collected therein is compared with the “baseline” information/data collected in analysis step 20 in order to determine any improvements in snoring activity of the patient resulting from implementation of the anti-snoring solutions provided in advice step 40. Depending on the results of such comparison, the patient is then provided with further advice and/or guidance at guide step 80 regarding changes or continuance of the anti-snoring solution(s) provided in advice step 40. It is thus to be appreciated that the general purpose of the guide step 80 is to empower users/snorers toward one or more of the following aspects: a) to use the anti-snoring devices in the correct manner. This is particularly important for devices that require a certain level of acceptance for a comfortable sleep, such as position correction devices; b) to (re)adjust some of the settings of the anti-snoring devices for improved comfort and/or for compensating (temporary) changes of the respiratory tract physiology, (re)adjustment function is enabled by for example the strap of a position correction device, a smaller/larger diameter for a nasal dilator, and the titration level of a mandibular advancement device; c) guidance/coaching for lifestyle changes that are known to decrease snoring related complaints, such as weight loss, breathing exercises; d) guidance/advice to use and/or change settings of anti-snoring devices by using the feedback from external factors, e.g. advising a nasal dilator coupled to the increasing risk of seasonal allergy complaints, and/or improving settings following medication intake, etc.; and e) to provide educational content for creating awareness about sleep disordered breathing and sleep disorders that users and/or their bed partners may face.

It is to be appreciated that the methods described herein may be implemented in by a computer program product including a non-transitory computer readable medium encoded with a computer program comprising program code (for implementing the methods).

It is to be appreciated that method 10 may be carried out, in-whole or in-part, through the use of various means without varying from the scope of the present invention. FIG. 2 shows, schematically, an arrangement of a system/device 100 (e.g., without limitation, a smart phone, a personal computer, etc.) which may be used to carry out method 10. Device 100 includes a processing device 102 having a suitable memory 104 associated therewith. Device 100 further includes one or more acoustic sensors 106 (e.g., microphones) in communication with processing device 102. Device 100 also includes an input/out device or devices 108 through which data may be sent to, or received from a patient or bed partner of the patient.

Accordingly, it is to be appreciated that embodiments of the invention assist a patient in not only finding an anti-snoring solution which is effective in regard to their particular snoring problem, but also ensures (as reasonably possible) that the user is properly/effectively utilizing such solution in a manner which provides the greatest benefit.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

1. A method for determining a personalized anti-snoring solution for a patient, the method comprising:
performing an analysis of a sleep period of the patient in which snoring by the patient is occurring;
determining an anti-snoring solution for the patient based on information obtained in the analysis;
advising the patient of the anti-snoring solution;
tracking a further sleep period of the patient in which the patient is employing the anti-snoring solution; and
advising the patient of changes to the anti-snoring solution based on a comparison of information obtained in the tracking with data obtained in the analysis.

2. The method of claim 1, wherein analyzing snoring by the patient comprises performing an acoustic analysis of the snoring by the patient.

3. The method of claim 2, wherein performing an acoustic analysis of the snoring by the patient comprises one or more of:
extracting snoring episodes from within a mixed acoustic signal;
classifying snoring intensity of the snoring episodes;
calculating the proportion of snoring episodes overnight;
classifying the snoring episodes sound amplitude; and
storing data resulting from the acoustic analysis.

4. The method of claim 2, wherein analyzing snoring by the patient comprises performing a spectral analysis of the snoring by the patient.

5. The method of claim 4, wherein analyzing snoring by the patient comprises storing data related to the sound of the snoring by the patient in a snoring sound library.

6. The method of claim 1, wherein analyzing snoring by the patient comprises performing a subjective assessment of snoring behavior by the patient.

7. The method of claim 6, wherein performing a subjective assessment comprises providing a number of questions to one or both of the patient and a bed partner of the patient and analyzing responses thereto.

8. The method of claim 1, wherein analyzing snoring by the patient comprises performing a self-assessment for determining a source of the snoring by the patient.

9. The method of claim 8, wherein performing a self-assessment comprises generating snoring sounds by closing and opening nostrils and mouth of the patient in order to identify the most likely source of the snoring by the patient.

10. A system for determining a personalized anti-snoring solution for a patient, the system comprising:
a processing device having a suitable memory associated therewith;
one or more acoustic sensors in communication with the processing device; and
an input/out device structured to receive information from and provide information to the patient,
wherein the processing device is programmed to:
utilize the one or more acoustic sensors in performing an analysis of snoring by the patient;
determine an anti-snoring solution for the patient based on information obtained in the analysis;
utilize the input/output device in advising the patient of the anti-snoring solution;
utilize the one or more sensors in tracking further snoring, by the patient in response to the patient employing the anti-snoring solution; and
utilize the input/output device in advising the patient of changes to the anti-snoring solution based on a comparison of information obtained in the tracking with data obtained in the analysis.

11. A computer program product including a non-transitory computer readable medium encoded with a computer program comprising program code for implementing the method of claim 1.
